(19) United States
(12) Patent Application Publication
(10) Pub. No.: US 2012/0215080 A1
(43) Pub. Date: Aug. 23, 2012

(54) APPARATUS, SYSTEM AND METHOD FOR TISSUE OXIMETRY

(75) Inventors: Chandan K. Sen, New Albany, OH (US); Savita Khanna, New Alrbany, OH (US); Sashwati Roy, New Albany, OH (US); Sangly P. Srinivas, Bloomington, IN (US)

(73) Assignee: The Ohio State University, Columbus, OH (US)

(21) Appl. No.: 13/351,850

(22) Filed: Jan. 17, 2012

Related U.S. Application Data
(63) Continuation of application No. 11/610,465, filed on Dec. 13, 2006, now abandoned.
(60) Provisional application No. 60/749,698, filed on Dec. 13, 2005.

(51) Int. Cl. A61B 5/1455; A61M 16/00

(52) U.S. Cl. 600/323; 128/204.23

(57) ABSTRACT

An apparatus, system and method for measuring oxygen concentration for exciting and detecting oxygen-sensitive fluorescence in biological tissues to detect oxygen levels (e.g., the partial pressure of oxygen).

### RELATED APPLICATION

The present application is being filed as a non-provisional patent application claiming the benefit under 35 U.S.C. §119(e) of U.S. Provisional Patent Application No. 60/749,698 filed on Dec. 13, 2005.

### FIELD

This application generally relates to the field of tissue oximetry, and more particularly, to tissue oximetry that involves using a fluorescent compound to measure oxygen concentration.

### BACKGROUND

Oxygen detection is a critical element of applied wound healing research and clinical wound management and is used for both diagnostic/prognostic and therapeutic purposes. Transcutaneous oximetry (hereinafter, TCOM) is a noninvasive process that directly measures the oxygen level of tissue beneath the skin. In particular, TCOM measures the amount of oxygen that reaches the skin through blood circulation.

In conventional TCOM, an area to be tested is first prepped (e.g., cleaned, shaved). A gel that conducts electrical impulses is then applied to the area. Adhesive sensors containing electrodes that can sense oxygen are applied to the area over the gel. Electrodes in the sensors heat the area below the skin to dilate the capillaries so oxygen can flow freely to the skin, which improves the reading. The readings are converted to an electrical current and the signal is displayed on a monitor and/or recorded.

Conventional TCOM, however, have many disadvantages. For example, conventional TCOM is based on electrochemical technology, wherein electrochemical detectors are used that consume oxygen while detecting it, which results in a risk of inaccurate results. Also, oxygen tension is read on the skin at the wound periphery, instead of the more preferable location of the actual wound bed. Furthermore, the electrochemical technology requires a relatively long time (e.g., about 45 minutes) to obtain an accurate oxygen measurement. Further still, unreliable measurements can occur in the presence of lower extremity edema, which is present in all patients with venous stasis ulcers, among other disorders.

Consequently, there is a need in the art for an improved apparatus, system and method for providing TCOM.

### SUMMARY

In view of the above, it is an exemplary aspect to provide an improved apparatus, system and method for measuring oxygen concentration using TCOM.

It is another exemplary aspect to provide an apparatus, system and method for exciting and detecting oxygen-sensitive fluorescence in biological tissues.

It is still another exemplary aspect to provide an apparatus, system and method for measuring oxygen-sensitive fluorescence using a frequency domain approach.

It is an exemplary aspect to provide a wound-implantable oxygen-sensitive fluorescence probe.

It is another exemplary aspect to provide an oxygen-sensitive fluorescence probe for performing TCOM.

It is yet another exemplary aspect to use feedback from tissue oximetry to control dosage during oxygen therapy.

### BRIEF DESCRIPTION OF THE DRAWINGS

The above aspects and additional aspects, features and advantages will become readily apparent by describing in detail exemplary embodiments thereof with reference to the attached drawings, wherein like reference numerals denote like elements, and:

FIG. 1 is a graph illustrating a phase delay between exemplary excitation and emission waveforms.

FIG. 2 is a graph illustrating phase delay measurements at various modulation frequencies for an exemplary pO₂-sensitive dye.

FIG. 3 is a graph illustrating the relationship between phase delay and pO₂ for an exemplary pO₂-sensitive dye.

FIG. 4 is a diagram of an exemplary system for measuring oxygen, according to an exemplary embodiment.

FIG. 5 is a graph illustrating N2-air transitions for an exemplary pO₂-sensitive dye.

FIG. 6 is a graph illustrating a typical phase-delay response to N2-air transitions.

FIG. 7 is a partial diagram of an exemplary device for measuring oxygen, according to an exemplary embodiment.

FIGS. 8A-8B are diagrams of an exemplary device for performing TCOM, according to an exemplary embodiment.

FIG. 9 is a diagram of a variation of the exemplary device of FIGS. 8A-8B, according to an exemplary embodiment.

FIG. 10 is a diagram of an exemplary excitation module and an exemplary emission module, according to an exemplary embodiment.

### DETAILED DESCRIPTION

While the general inventive concept is susceptible of embodiment in many different forms, there are shown in the drawings and will be described herein in detail specific embodiments thereof with the understanding that the present disclosure is to be considered as an exemplification of the principles of the general inventive concept. Accordingly, the general inventive concept is not intended to be limited to the specific embodiments illustrated herein.

According to an exemplary embodiment, a system 100 for measuring a partial pressure of oxygen (pO₂) is provided. The system 100 is based on oxygen-sensitivity of fluorescence of certain dyes. These dyes undergo modification (i.e., collisional quenching) in their excited state by molecular oxygen. In particular, if the excited dye encounters an oxygen molecule, excess energy is transferred to the oxygen molecule in a non-radiative transfer, thereby decreasing or quenching the fluorescence of the dye. The degree of quenching correlates to the level of oxygen concentration or the pO₂ in the oxygen-containing media (e.g., biological tissue). As a result, an increase in pO₂ decreases fluorescence intensity and lifetime with respect to the dye. Similarly, an increase in fluorescence intensity and lifetime with respect to the dye corresponds to a decrease in pO₂.

The emitted fluorescence of the dye is quantitatively related to the pO₂ by the Stern-Volmer equation, i.e., Equation 1.

$$\frac{{F}_{0}}{F} = 1 + {K}_{SV} {p O}_{2} \qquad ( Equation 1 )$$

where F₀ is the fluorescence when the pO₂=0, where F is the measured fluorescence at pO₂, and where K_{SV} is the Stern-Volmer constant. Thus, F₀ is the unquenched fluorescence intensity and F is the fluorescence intensity for the pO₂. Accordingly, if F₀ and F are known, the pO₂ can be determined.

Since the steady state fluorescence of the dye is dependent on its concentration, measuring an intrinsic parameter of the dye such as its fluorescence lifetime is useful. The fluorescence lifetime of the dye is quantitatively related to the pO₂ by an alternative form of the Stern-Volmer equation, i.e., Equation 2.

$$\frac{{F}_{0}}{F} = \frac{{τ}_{0}}{τ} = 1 + {K}_{SV} {pO}_{2} \qquad ( Equation 2 )$$

where τ₀ is the lifetime when pO₂=0, where τ is the measured lifetime at pO₂, and where K_{SV} is the Stern-Volmer constant. Thus, τ₀ is the unquenched lifetime and τ is the lifetime for the pO₂. Accordingly, if τ₀ and τ are known, the pO₂ can be determined.

A direct approach for measuring the lifetime of the oxygen-sensitive dyes is to follow the rate of fluorescence decay in response to a pulse excitation. This time-domain approach, however, does not result in faster acquisition of pO₂ samples.

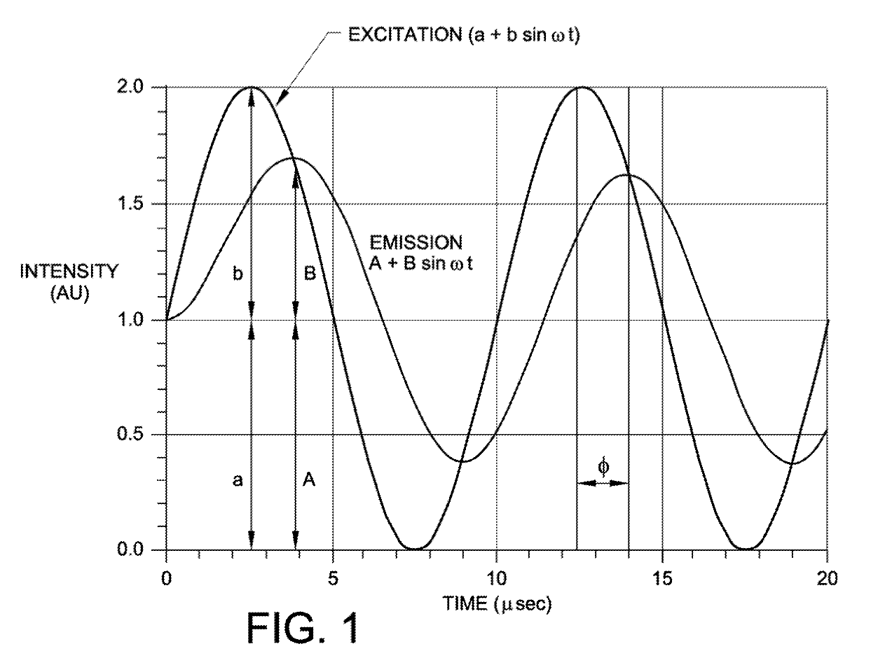

This problem of slow acquisition times is avoided by the frequency-domain approach of the system 100. Accordingly, in the system 100, changes in fluorescence lifetimes appear as changes in the phase delay of an emission wave when the excitation is via an intensity modulated sine wave, as shown in FIG. 1. The phase delay is related to the fluorescence lifetime of the dye by Equations 3-5.

tan Φ=ωτ (Equation 3)

where Φ is the phase delay, where ω is the angular frequency (expressed in radians in per second), and where τ is the fluorescence lifetime of the dye for the pO₂.

ω=2πf (Equation 4)

where ω is the angular frequency (expressed in radians in per second), and where f is the frequency (expressed in cycles per second).

$$M = \frac{1}{\sqrt{( 1 + {ω}^{2} {τ}^{2} )}} \qquad ( Equation 5 )$$

where M is Amplitude modulation, where ω is the angular frequency (expressed in radians in per second), and where τ is the fluorescence lifetime of the dye for the pO₂.

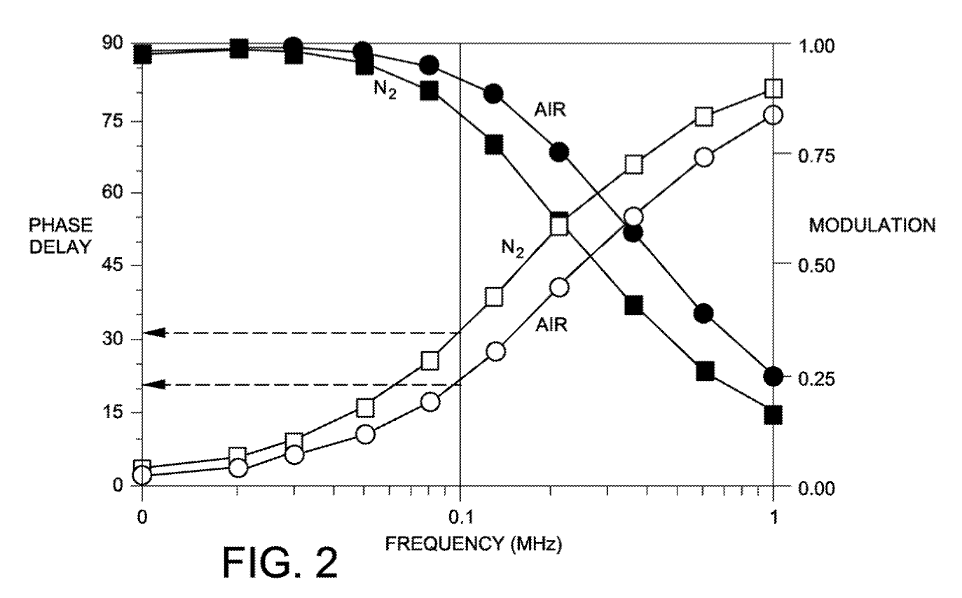

It will be appreciated that any suitable oxygen-sensitive (e.g., pO₂-sensitive) dyes can be used. For example, Tris(1,10 phenatroline)ruthenium (II) (hereinafter, Ru[Phen]) is one such dye. Ru[Phen] is a fluorescent dye with an excitation wavelength (λₑₓ) of 460 nm and an emission wavelength (λₑₘ) greater than 600 nm. Several phase delay measurements were obtained using a commercial lifetime fluorometer at various modulation frequencies for Ru[Phen], as shown in FIG. 2.

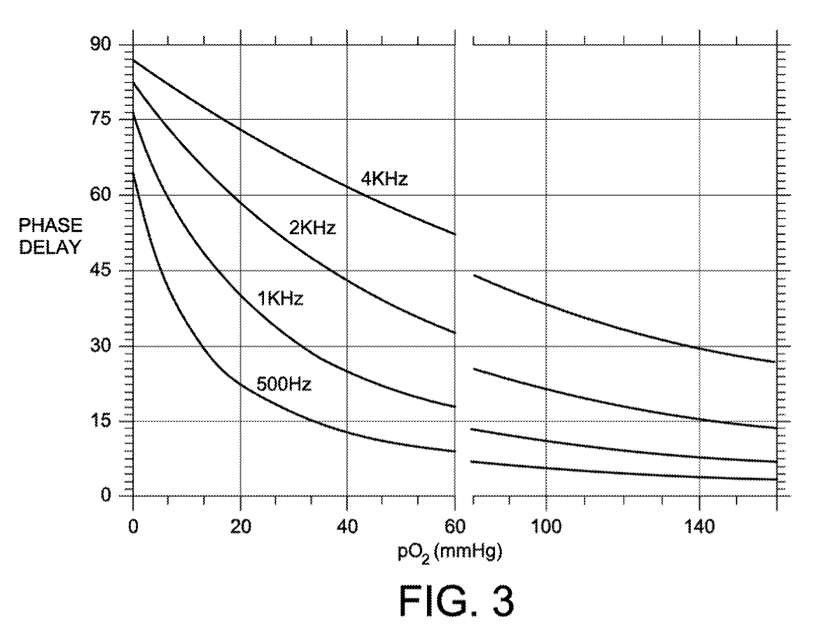

Pd-meso-tetra (4-carboxyphenyl)porphyrin (hereinafter, Pd-porphyrin), which has been used in human studies, is another exemplary dye. Pd-porphyrin is a phosphorescent dye with an excitation wavelength (λₑₓ) of 523 nm and an emission wavelength (λₑₘ) greater than 600 nm. A phase-delay vs. pO₂ plot for Pd-porphyrin, which has a long lifetime, is shown in FIG. 3. The plot was simulated assuming K_{SV}=300 mmHg-1 sec⁻¹ and τ₀=640 ms. As can be seen in FIG. 3, the Pd-porphyrin exhibits a high sensitivity for pO₂ in the range of 0-60 mmHg.

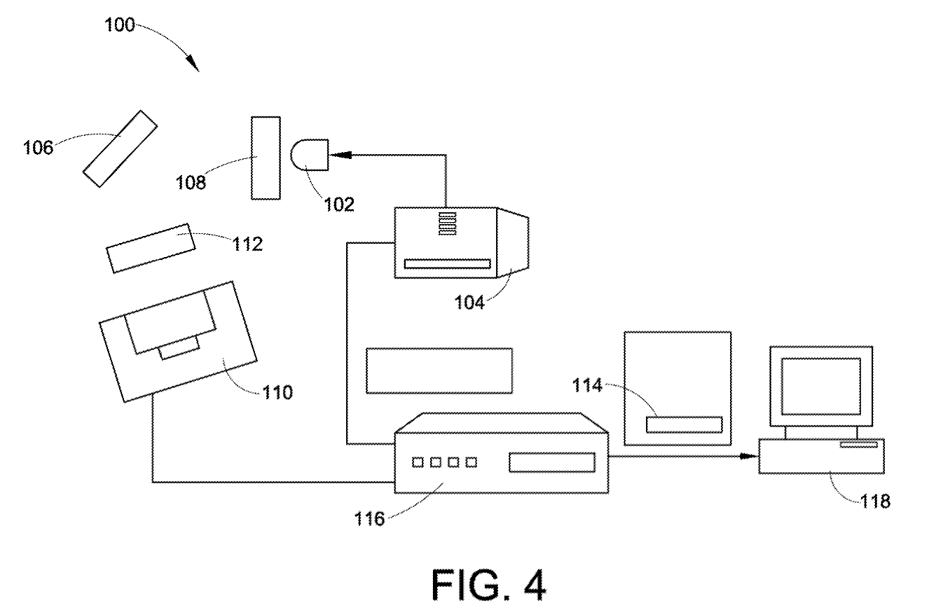

The exemplary system 100 is shown in FIG. 4. The system 100 includes, for example, an excitation source 102 (e.g., a light source) and a function generator 104. In one exemplary embodiment, the excitation source 102 is a blue LED. In another exemplary embodiment, the excitation source 102 is a green LED. Light from the excitation source 102 is intensity modulated as a sine wave by the function generator 104. In an exemplary embodiment, the sine wave is 6 volts peak-to-peak. In an exemplary embodiment, the light from the excitation source 102 is intensity modulated at 1 KHz. In another exemplary embodiment, the light from the excitation source 102 is intensity modulated at 100 KHz.

The modulated output of the excitation source 102 (i.e., an excitation wave) is directed to the surface or other area of a media 106 to be measured. In an exemplary embodiment, the media 106 is a polymeric film containing a pO₂-sensitive dye. The dye can be Ru[Phen], Pd-porphyrin or any other suitable dye. In another exemplary embodiment, the media 106 is a probe with a portion (e.g., a tip) of the probe containing the dye.

A filter 108 is disposed between the excitation source 102 and the media 106 to limit the excitation wavelength of the modulated output of the excitation source 102. In an exemplary embodiment, the peak excitation wavelength is 460 nm. In another exemplary embodiment, the peak excitation wavelength is 530±40 nm.

A fluorescence emission (i.e., an emission wave) leaves the media 106 at an angle (e.g., of about 60 degrees) relative to an excitation axis. A detector 110 detects the fluorescence emission from the media 106. In an exemplary embodiment, the detector 110 is a high speed avalanche photodiode.

Another filter 112 is disposed between the media 106 and the detector 110 to limit the emission wavelength. In an exemplary embodiment, the peak emission wavelength is greater than 600 nm.

A phase delay 114 between the excitation and emission waves is measured by a phase detector 116. In an exemplary embodiment, the phase detector 116 is a lock-in amplifier having a bandwidth of 120 KHz. The phase delay 114 is then transmitted to a computer 118, for example, at 1 KHz and at a resolution of 16 bits.

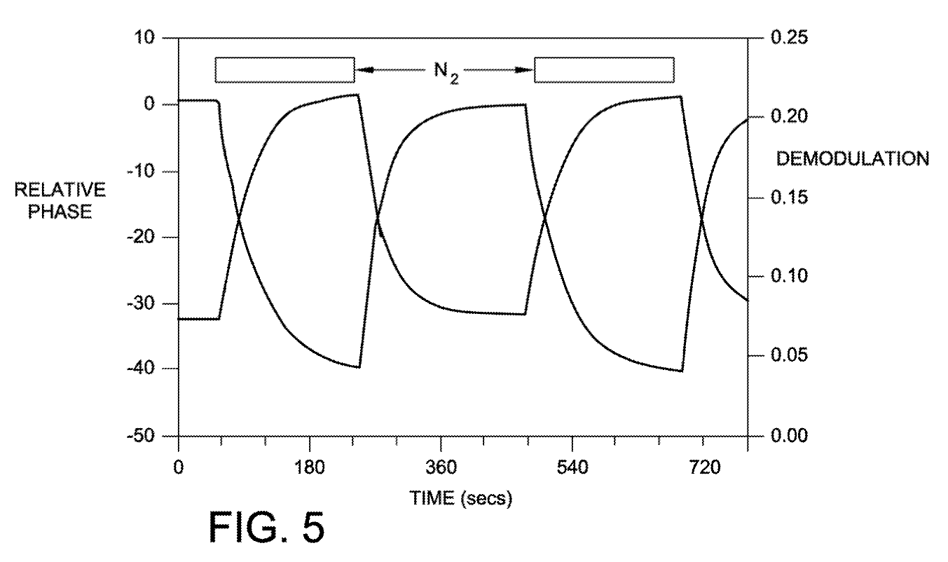

Exposure of the media 106 to an oxygen-deprived environment (e.g., by subjecting the media 106 to an N₂ stream) leads to a rapid increase in both the phase delay 114 and an intensity of fluorescence consistent with a decrease in the extent of quenching by the loss of the oxygen. The transitions between the media 106, which contains the Ru[Phen] dye, being exposed to air (containing oxygen) and N₂ (without oxygen) are illustrated in FIG. 5.

Each time the N₂ stream ends, the diffusion of oxygen into the media 106 begins immediately and results in the phase delay 114 and the intensity of fluorescence returning to their original values, which is consistent with an increase in quenching owing to the elevated oxygen levels in the media 106.

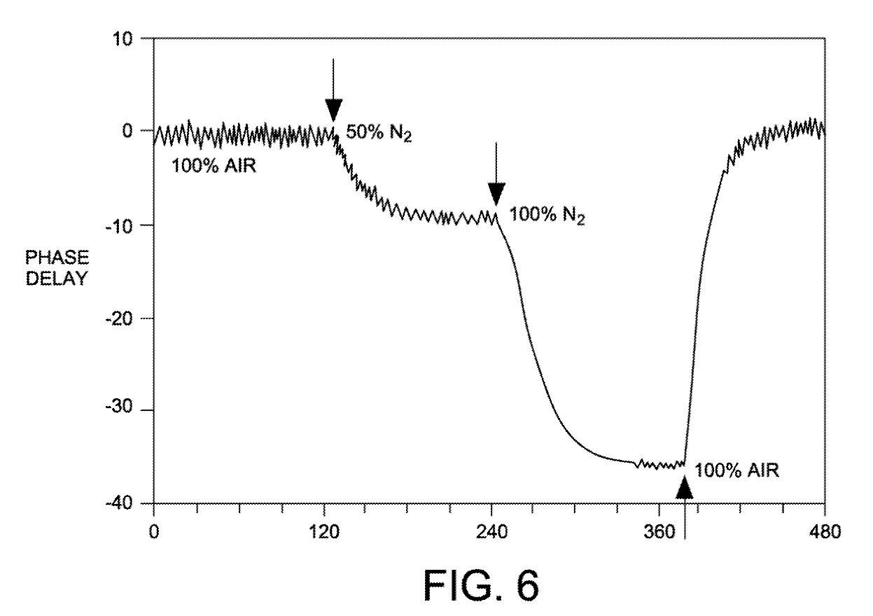

A typical phase-delay response resulting from N₂-air transitions is illustrated in FIG. 6. The changes in the phase delay 114 and demodulation can be correlated to the pO₂ in the N₂-air mixture levels using, for example, the Stern-Volmer equations described above.

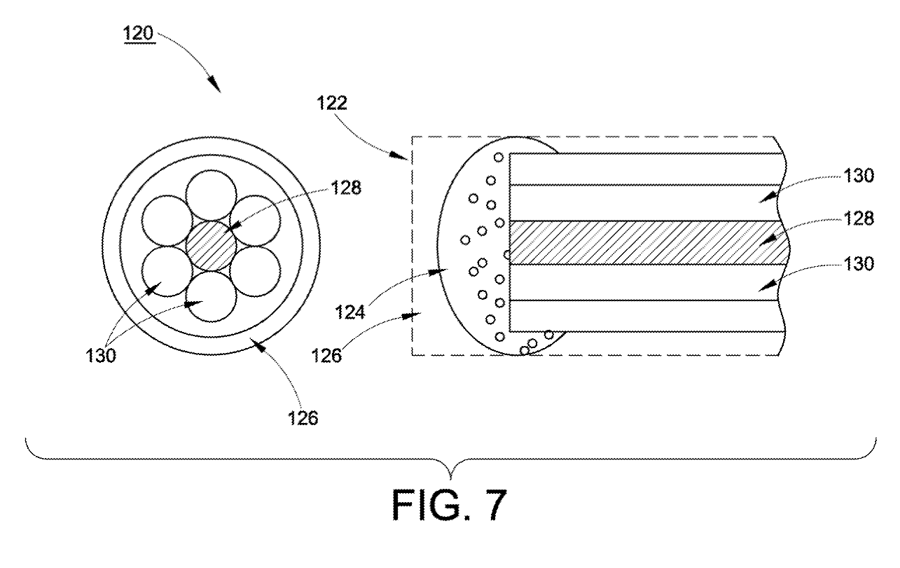

In view of the exemplary system 100 described above, various apparatuses and methods can also be used for measuring pO₂ based on oxygen-sensitive dyes. An exemplary device 120 (e.g., a probe) for measuring pO₂, according to an exemplary embodiment, is shown in FIG. 7.

The device 120 includes, for example, a tip 122 or other portion that contains a pO₂-sensitive fluorescence dye (e.g., in film or tablet form). In an exemplary embodiment, a sensor film 124 containing the dye is located in the tip 122. In the sensor film 124 the dye is bound to silica microparticles in silicone rubber. The device 120 also includes, for example, a bifurcated fiber optic bundle forming a Y-end (not shown). One arm of the Y-end is connected to an excitation module which is described below. The other arm of the Y-end is connected to an emission module which is described below.

The position of the tip 122 of the device 120 determines the locale from which the pO₂ is sensed. The device 120 can be implanted into the actual wound bed for more accurate readings.

A Silastic (a registered trademark of Dow Corning Corp.) tubing 126 surrounds the tip 122 and the fiber optic bundle. The use of the Silastic tubing 126 permits facile oxygen flux into the embedded oxygen-sensitive dye at the tip 122 of the device 120.

The bifurcated fiber optic bundle has an excitation fiber 128 at its core. Several emission fibers 130 encircle the excitation fiber 128.

Because the device 120 is intended for localization in the wound bed, the sensor film 124 is likely to undergo fouling. Accordingly, periodic replacement of the sensor film 124 may be necessary. To facilitate the replacement of the sensor film 124, it is easy to disconnect the tip 122 from the device 120 and remove the sensor film 124 at the end of the fiber optic bundle.

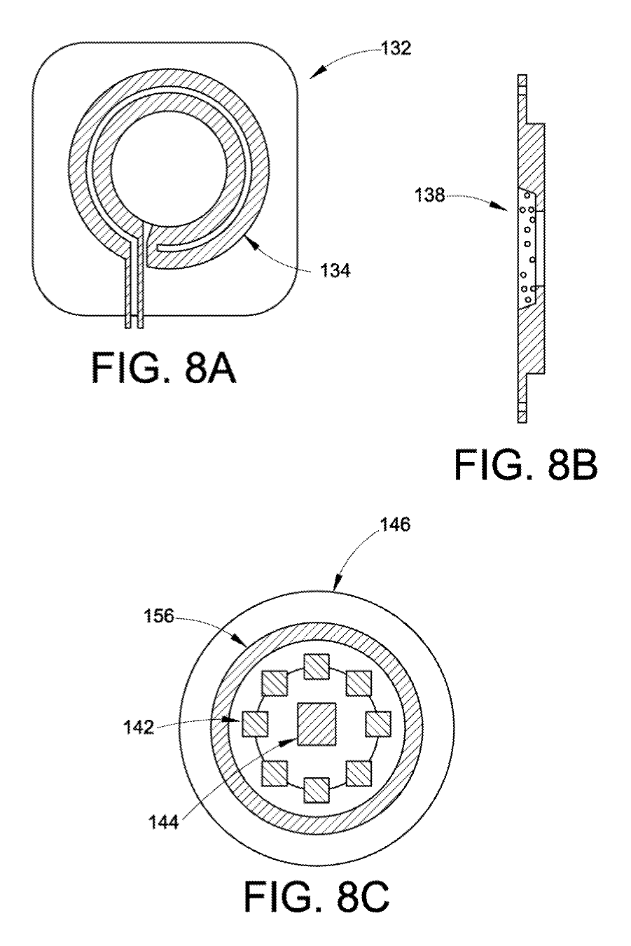

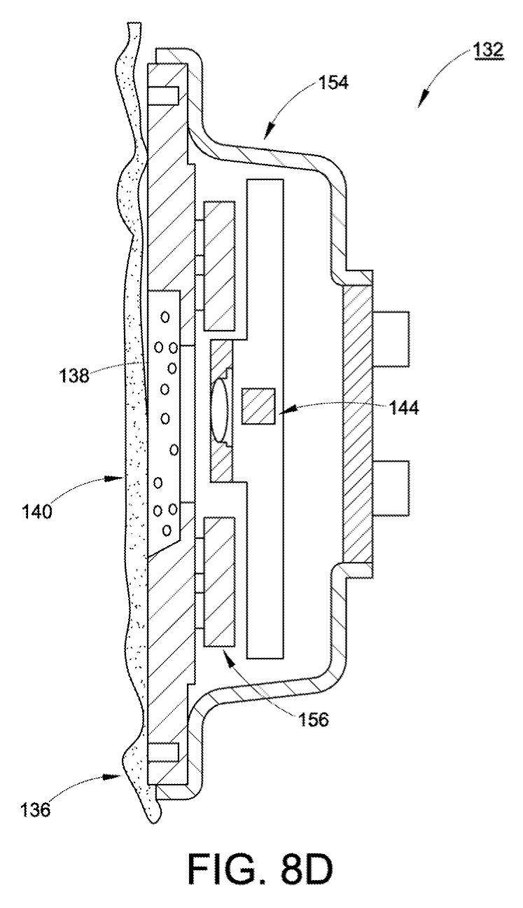

An exemplary device 132 (e.g., a probe) for performing TCOM, according to an exemplary embodiment, is shown in FIGS. 8A-8B. The device 132 includes a heating element 134 (e.g., a platinum electrode) for raising the temperature of the skin 136 under a sensor film 138 of the device 132. In an exemplary embodiment, the skin 136 under the sensor film 138 is raised to 44° C. by the heating element 134. The increased skin temperature results in elevated perfusion to the area under the sensor film 138. As this hyperfusion overwhelms the local demand, oxygen in the blood diffuses into a sampling volume 140 under the device 132.

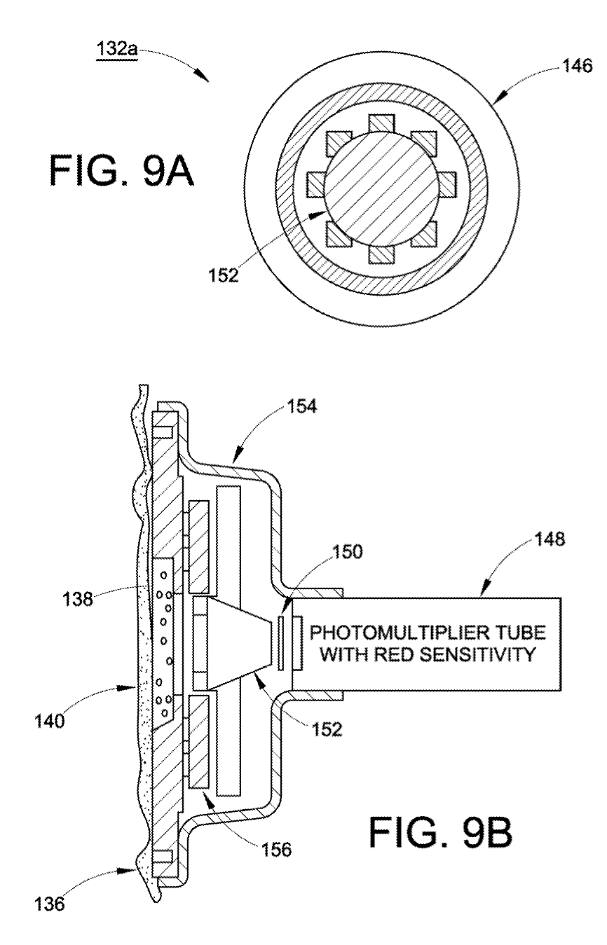

A change in the pO₂ in the sampling volume 140 is then sensed through changes in fluorescence lifetime of an oxygen-sensitive dye embedded in the sensor film 138. Such changes are measured by using an excitation source 142 (e.g., a blue LED) and detecting an emission using a detector 144, wherein the excitation source 142 and the detector 144 are held together by a detector plate 146. In an exemplary embodiment, the detector 144 is an avalanche photodiode, as shown in FIGS. 8A-8B. In another exemplary embodiment, a device 132a includes the detector 144 is a head-on photomultiplier tube 148, and includes a filter 150 and a fiber optic plate 152, as shown in FIG. 9.

The components of the device 132, 132a are held hermetically sealed in an enclosure 154. In an exemplary embodiment, the enclosure 154 is formed so as to facilitate replacement of the sensor film 138. The enclosure 154 can be light-proof and/or made of a polymeric material. The enclosure 154 can include an insulator 156 that thermally and/or electrically insulates the device 133 and 132.

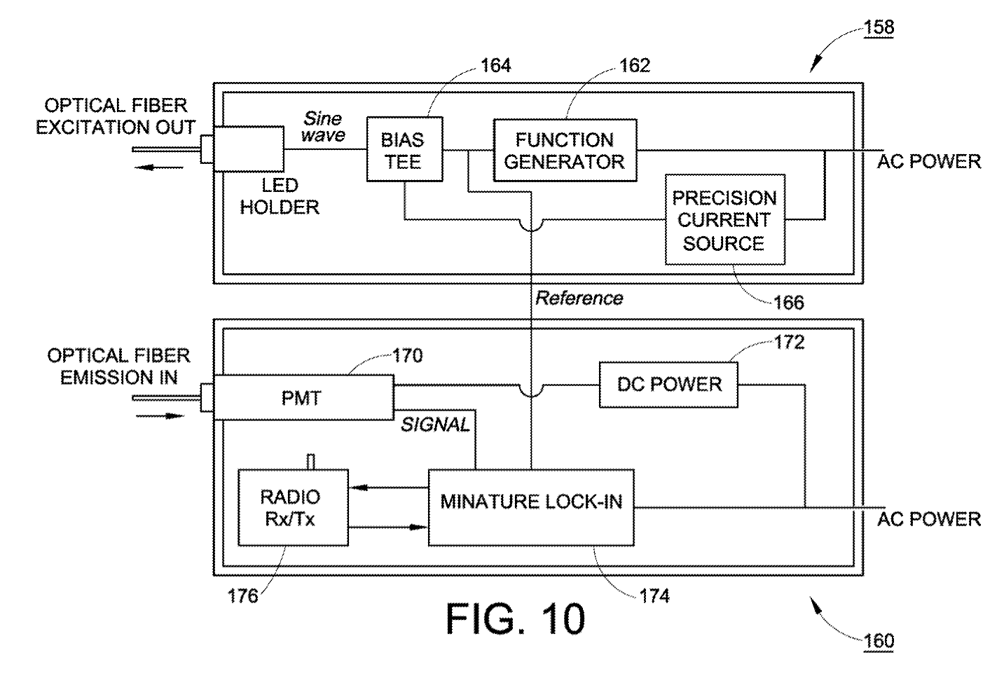

The devices (e.g., devices 120, 132, 132a) are connected to an excitation module 158 and an emission module 160 to record the pO₂. See FIG. 10. The structure of the excitation module 158 is similar for both the device 120 and the device 132/132a. For the wound implantable device (i.e., device 120), the excitation module 158 produces the intensity-modulated excitation light output which is connected to the excitation arm of the tip 122 of the device 120. The excitation light can be, for example, a blue or green LED. The modulation is produced by a sine-wave generator 162 (i.e., function generator) and frequencies between 4-200 KHz. The output of the function generator 162 is connected to the LED through a bias-tee 164. Power to the LED injected through the bias-tee 164 is derived from a stable and precise current source 166. The current source 166 and the function generator 162 can be controlled through a radio telemetric receiver and transmitter (not shown) in the excitation module 158. In the case of the TCOM devices (i.e., devices 132 and 132a), the output of the bias-tee 164 is fed to the LEDs on the detector plate 146.

The structure of the emission module 160 is similar for both the device 120 and the device 132/132a. In the case of the wound implantable device (i.e., the device 120), the emission module 160 receives the fluorescence emission through one of the arms of the fiber optic bundle. This emission can be detected by a photomultiplier 170 with a built-in high-voltage source 172 and trans-impedance amplifier 174. The phase delay in the emission relative to the excitation can be detected by the dual phase lock-in amplifier 174. The reference for the lock-in is synched to the sine wave generator 162 of the excitation module 158.

The analog outputs of the lock-in phase delay and magnitude are sampled at a resolution of 16 bits and 1 sample per second. The digital output can then be sent to a remote computer via an embedded radio-telemetric receiver and transmitter 176. For the TCOM device, the trans-impedance amplifier 174 will be held close to the photomultiplier tube 148 or the avalanche photodiode 144, which will be part of the sensor package itself, to prevent contamination of low-level signals. The excitation module 158 and the emission module 160 facilitate high speed wound/bed oximetry.

In one exemplary embodiment, software monitors the outputs of the lock-in amplifier 174 and provides feedback control signals to a control unit of a hyperbaric chamber. In this manner, the oximetric feedback is used so that the hyperbaric chamber is automatically pressurized to the prescribed pO₂. Accordingly, the oximetric feedback allows the oxygen therapy to be much more personalized.

Other exemplary functions of the software include: (1) telemetric setting of the function generator 162 and the current source 166; (2) telemetric setting of the lock-in amplifier 174 in real time; (3) providing a user interface for parameter settings and remote monitoring of pO₂ and skin temperature; and (4) providing a database for archiving patient-dependent information in a secure manner.

The above description of specific embodiments has been given by way of example. From the disclosure given, those skilled in the art will not only understand the general inventive concept and its attendant advantages, but will also find apparent various changes and modifications to the structures and methods disclosed. It is sought, therefore, to cover all such changes and modifications as fall within the spirit and scope of the general inventive concept, as defined herein, and equivalents thereof. Thus, the embodiments described in the specification are only exemplary or preferred and are not intended to limit the terms of the claims in any way. The terms in the claims have all of their broad ordinary meanings and are not limited in any way or by any descriptions of these exemplary embodiments.

1.-18. (canceled)

19. An apparatus for measuring an oxygen level, the apparatus comprising:
a heating element for raising the temperature of skin at a site to be measured;
a sensor unit including an oxygen-sensitive dye;
an excitation source for generating excitation light in the form of an intensity modulated sine wave;
a detector for detecting an emission from the dye in response to the excitation light contacting the dye;
a phase detector for detecting a phase delay between the excitation light and the emission; and
a computing device determining the oxygen level at the site based on the phase delay, and providing a signal to a control unit for adjusting the oxygen level at the site to achieve a prescribed oxygen level at the site.

20. The apparatus of claim 19, wherein the signal provided to the control unit is a feedback control signal based on the oxygen level at the site.

21. The apparatus of claim 19, wherein the excitation source comprises at least one light emitting diode.

22. The apparatus of claim 19, wherein the excitation light is one of blue and green.

23. The apparatus of claim 19, wherein the detector comprises an avalanche photodiode.

24. The apparatus of claim 19, wherein the detector comprises a photomultiplier.

25. The apparatus of claim 19, wherein the phase detector comprises a dual phase lock-in amplifier.

26. The apparatus of claim 19, wherein:
the phase delay is related to a fluorescence lifetime of the dye as:
tan Φ=ωτ, where
Φ is the phase delay,
ω is an angular frequency expressed as radians per second, and
τ is the fluorescence lifetime of the dye.

27. The apparatus of claim 19, wherein the excitation source includes:
a function generator generating the intensity modulated sine wave.

28. The apparatus of claim 19, wherein the oxygen level is displayed by the computer.

29. The apparatus of claim 19, wherein:
the computing device compares the determined oxygen level with the prescribed oxygen level; and
the computing device provides the signal to the control unit based on the comparison.

30. A method of measuring an oxygen level at a site, the method comprising:
generating an excitation wave as an intensity modulated light forming a sine wave;
focusing the excitation wave on an oxygen-sensitive dye;
detecting an emission wave emitted in response to the excitation wave contacting the dye;
determining a phase delay between the excitation wave and the emission wave;
determining the oxygen level at the site based on the phase delay; and
providing a signal to a control unit, based on the phase delay, for adjusting the oxygen level at the site to achieve a prescribed oxygen level at the site.

31. The method of claim 30, further comprising locating the dye inside a wound prior to focusing the excitation wave on the dye.

32. The method of claim 30, wherein the step of generating includes:
generating one of blue and green light.

33. The method of claim 30, wherein the step of generating includes:
generating the sine wave having a frequency between 4 and 200 kHz.

34. The method of claim 30, wherein the step of providing a signal includes:
providing a feedback control signal to a control unit of a chamber.

35. The method of claim 34, further including:
determining oximetric feedback from the feedback control signal; and
adjusting the oxygen level at the site to achieve the prescribed oxygen level at the site based on the oximetric feedback.

36. The method of claim 30, further including:
comparing the determined oxygen level with the prescribed oxygen level, and
providing the signal to the control unit based on the comparison.

37. The method of claim 30, further including:
remotely monitoring the oxygen level.

38. An apparatus for measuring an oxygen level, the apparatus comprising:
a heating element for raising the temperature of skin at a site to be measured;
a sensor unit including an oxygen-sensitive dye;
means for generating excitation light in the form of an intensity modulated sine wave;
a detector for detecting an emission from the dye in response to the excitation light contacting the dye;
a phase detector for detecting a phase delay between the excitation light and the emission;
a computing device determining the oxygen level based on the phase delay; and
means for adjusting the oxygen level at the site to achieve a prescribed oxygen level at the site.

39. The apparatus for measuring an oxygen level as set forth in claim 38, wherein:
the means for generating is light emitting diode.

40. The apparatus for measuring an oxygen level as set forth in claim 38, wherein:
the means for detecting is an avalanche photodiode.

41. The apparatus for measuring an oxygen level as set forth in claim 38, wherein:
the means for adjusting adjusts the oxygen level at the site based on the determined oxygen level.
